(19) United States
(12) Patent Application Publication
(10) Pub. No.: US 2025/0375196 A1
(43) Pub. Date: Dec. 11, 2025

(54) SUTURE DEVICE

(71) Applicant: Mattia Glauber, Milano MI (IT)

(72) Inventor: Mattia Glauber, Milano MI (IT)

(21) Appl. No.: 18/879,176

(22) PCT Filed: Jun. 27, 2023
(86) PCT No.: PCT/IB2023/056610

§ 371 Date: Dec. 26, 2024

(30) Foreign Application Priority Data
Jun. 27, 2022 (IT) 102022000013501

(51) Int. Cl. A61B 17/04; A61B 17/00; A61B 17/06

(52) U.S. Cl. CPC A61B 17/0469; A61B 17/06066; A61B 2017/00946; A61B 2017/0608

(57) ABSTRACT

The present invention relates to a suture device, in particular a suture passer and/or needle pusher device for facilitating suture operation. Said device is provided with a hollow tube, a handpiece connected to the proximal end of the hollow tube, the handpiece comprising an actuator, and a chuck disposed within the hollow tube. Advantageously, the device is also provided with at least one stop means for facilitating the needle loading operation and, at the same time, ensuring the safety of the device both in use and at rest.

### FIELD OF THE INVENTION

The present invention relates generally to a surgical suture device and in particular to a suture-passer and/or needle-pusher device for passing the needle and thus the suture thread through a tissue and facilitating the suturing operation.

### BACKGROUND ART

The use of suture devices is widespread in medical practice.

The term “suture devices” or “devices of suture” in the context of the present disclosure generally refers to surgical instruments used to make sutures. These instruments, also referred to as “suturers” may vary in shape and size, may be sterile or non-sterile devices, are generally reusable and are usually made of stainless steel. Non-limiting examples of suture devices are: agraphes applicator, suture thread guide, ligator (umbilical, for hemorrhoids, for oesophageal vessels, etc.), suture passer for arthroscopic surgery, suture positioner, thread pusher and/or needle pusher, etc.

Generally, the suture thread is attached to, or threaded into, a suture needle that is passed through the tissue to be sutured. However, when the surgical site is difficult to access and/or it is necessary to precisely control the suture, suture-passing devices (or needle-pushers) are often used that can precisely direct and guide the needle through tissues.

Suture-passers and/or needle-pusher devices known in the art generally comprise a rod with a handle at one end and a holding system at the other end, into which the suture thread is inserted. For example, WO2010/144992 describes a device for use in arthroscopic rotator cuff repair comprising an awl having a handle and a shaft for transmitting force to a distal hooked portion having a sharp tip capable of penetrating bone tissue. The tip can also be fitted with an eyelet configured to receive a suture thread. Although the hook configuration of the shaft can be advantageous in directing the suture wire, the presence of such a sharp tip, and the fact that the tip is always exposed, increases the risk of accidental tissue injury during surgery. Furthermore, with this type of device, control of the movement and dosage of force is solely left to the operator.

More complex suture-passing devices are also known in the art that feature a needle-holding system with a “clamp” configuration, such as, for example, endoscopic suture devices marketed under the name “SILS Stitch” (Covidien) and “Endo Stitch” (Medtronic) in which the needle is passed from one arm of the clamp to the other through two aligned holes. However, these instruments involve the use of special cartridges to load the needle and/or suture thread and require a great deal of experience on the part of the operator. In fact, if the operator operates the device when the plier arms are not in a neutral condition, i.e. the position that supports the needle, there is a risk of irreversibly bending the needle or even losing alignment with the hole on the other arm of the pliers, compromising the suture and forcing the operator to retrieve the needle manually.

It is therefore an object of the present invention to provide a suture device, in particular a suture-passing device, which is improved over devices known in the art, i.e. which does not have the aforementioned drawbacks. For example, an object of the invention is to provide a suture-passing device which allows easy loading of the needle and thread and is also easy and safe to handle.

### SUMMARY

To this end, a suture-passing device is provided which allows a front loading of the needle and thread and provides at least one safety system from among those better defined below. Therefore, an object of the present invention is a suture system, in particular a suture passing and/or needle passing device, the essential features of which are as defined in the appended claim 1. Other ancillary technical features are the subject matter of the dependent claims.

As will be apparent to a person skilled in the art, the suture passing and/or needle passing device of the present invention provides fine control of the movement, averts inadvertent dislocation of the needle, and allows easy loading of the needle and/or suture thread. Other aspects, features and advantages of the system and anchoring method of the present invention will be apparent from the detailed description below.

### BRIEF DESCRIPTION OF THE DRAWINGS

By way of example, an embodiment of the suture-passing and/or needle-pusher device of the invention is shown, with reference to the appended drawings, wherein:

FIG. 1 is a sectional view of a suture-passing device according to an embodiment of the invention

FIG. 2 is an enlargement of the section of the suture-passing device, represented in FIG. 1, at the handpiece of the device;

FIGS. 3a, 3b represent a perspective view of a chuck inside the tube of the suture passer device, according to an embodiment of the invention, and of a suture needle preloaded with a thread;

FIGS. 4a, 4b represent a perspective view of the chuck inside the tube of the suture passer device comprising a hook apex according to another embodiment of the invention, and of the suture needle pre-loaded with a thread;

FIGS. 5a-5d represent a perspective view of a chuck within the tube of the suture passer device comprising end flaps according to a further embodiment of the invention, and of the suture needle pre-loaded with a thread;

FIG. 6 is a perspective view in transparency of the end portion of the tube of the suture passing device implementing the chuck and needle shown in FIGS. 3a, 3b;

FIGS. 7a, 7b are a perspective view in transparency of the tube end portion of the suture passing device implementing the chuck and needle shown in FIGS. 4a, 4b, wherein the tube includes a groove on its outer curvature;

FIGS. 8a, 8b represent a perspective view in transparency of the tube end portion of the suture pass device implementing the chuck and needle shown in FIGS. 4a, 4b, wherein the tube comprising a groove on its internal curvature;

FIGS. 9a, 9b represent a perspective view in transparency of the tubing end portion of the suture passing device implementing the chuck and needle shown in FIGS. 5a-5d, wherein the tubing includes a groove on its internal curvature.

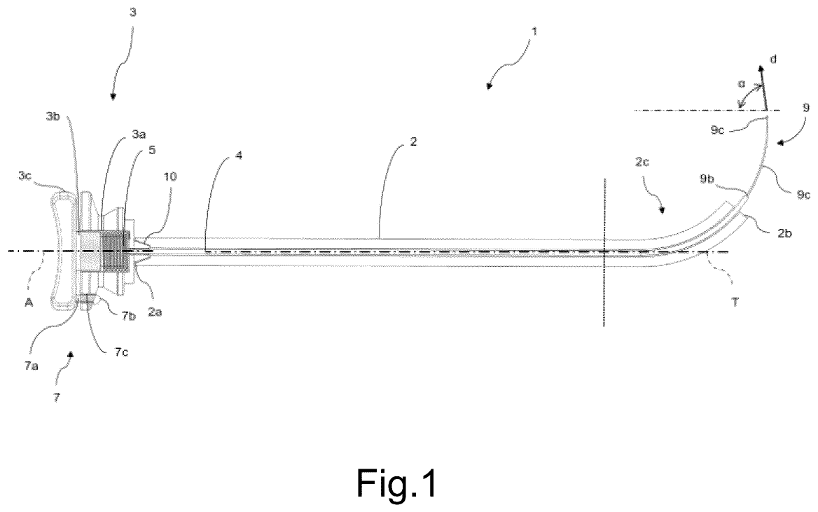

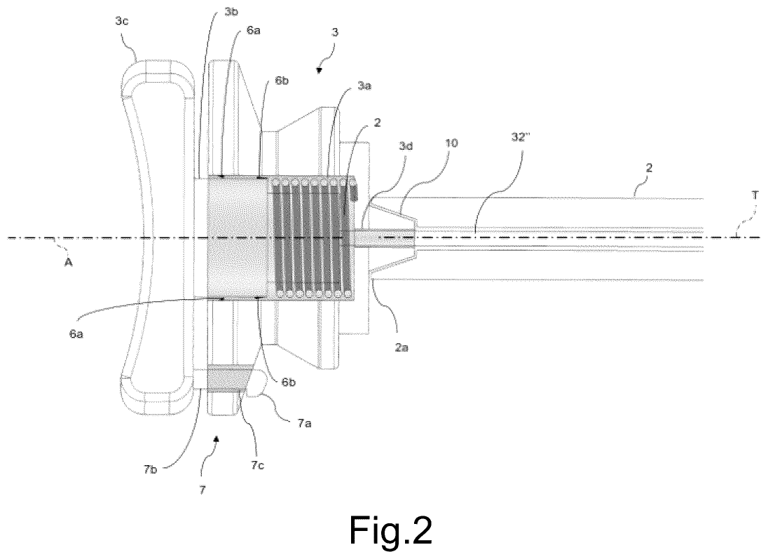

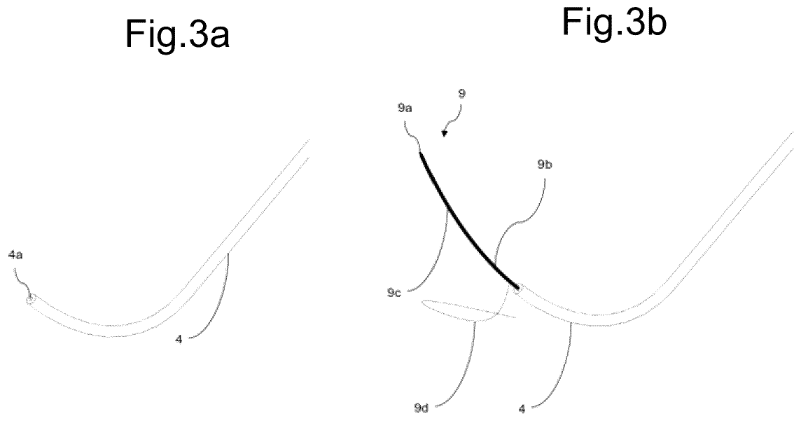

The thicknesses and dimensions depicted in the above Figures should be understood as purely illustrative, are generally magnified and not necessarily shown in proportion.

### DETAILED DESCRIPTION OF PREFERRED EMBODIMENTS

Various embodiments and variations of the suture-passer and/or needle-pusher device according to the present invention will be described below, and this with reference to the figures introduced above. Similar components are denoted in the various figures with the same numerical reference.

In the following detailed description, further embodiments and variants with respect to embodiments and variants already dealt with in the description will be illustrated only with respect to the differences with what has already been disclosed.

Furthermore, the various embodiments and variants described below can all be used in combination, where compatible.

In its simplest embodiment, the suture-passer and/or needle-pusher device 1 of the present disclosure comprises a hollow tube 2 having a rectilinear proximal tube portion defining a longitudinal tube axis T, a handpiece 3, an actuator 3b defining an actuation axis A and a chuck 4.

In particular, the hollow tube 2 has a proximal end 2a and a distal end 2b. The handpiece 3 is reversibly connected to the proximal end of the hollow tube 2, and the hollow tube 2 extends coaxially from the handpiece along a distal direction. Said handpiece 4 is located within the hollow tube 2 and sized to have a diameter smaller than the lumen diameter defined by the hollow tube 2 and a length equal to or greater than the length of the hollow tube 2.

In the context of the present disclosure, the Italian term “mandrino”, corresponding to the English term “chuck”, refers to a thin metal rod or wire configured to be inserted into a hollow cylindrical medical device. According to the present invention, the chuck 4 is made of any material suitable for the application described herein, for example steel or polymeric material with high mechanical properties.

The hollow tube 2 comprises a curved distal portion 2c extending from said proximal straight tube portion to said distal end 2b of the hollow tube and defining a housing for a suture needle 9, wherein the term “suture needle 9” preferably means a curved piercing needle suitable for insertion into a biological tissue and made of a biocompatible material, which is already widely known in the state of the art concerning biomedical devices. In particular, the distal end 2b is configured to receive and pass a suture needle 9 into said curved distal tube portion 2c.

Said curved distal portion 2c is characterised by a profile curved with respect to the tube axis T so that the distal end 2b of the hollow tube 2 is oriented transversely with respect to the tube axis T. According to any one embodiment of the present invention, the curved profile of the distal portion 2c is a hook profile. In particular, said curved distal tube portion 2c describes an arc of circumference subtended by a first angle at the centre of not less than 45°, preferably not less than 60°, more preferably not less than 90°, and is configured to accommodate within it a curved suture needle also describing a second arc of circumference not longer than said first arc of circumference, said second arc of circumference having the same radius as said first arc of circumference so as to facilitate insertion of the needle within the curved distal portion 2c.

The flexible nature of the chuck 4 allows the chuck 4 to follow the curved profile of the curved distal portion 2c of the hollow tube 2 in which it is inserted.

According to any of the embodiments of the present invention, said curved distal portion 2c is made of rigid, non-deformable material, while the remaining portion of the hollow tube 2 not forming part of the curved distal portion 2c can be made of plastically deformable material, so as to allow the bending of the hollow tube 2 for the purpose of varying the orientation of the hollow tube 2 and the check 4 therein.

Preferably, as can also be appreciated from the drawings, the tube end 2b is flat and blunt so as to minimise the risk of unintentional injury.

The device may comprise a suture needle 9 comprising a curved body 9c and defining two ends, said two ends being a piercing tip (9a) and a needle base (9b). Prior to use, said needle 9 is coupled to a medical-grade suture thread 9d. Preferably, needles with pre-mounted thread are used.

In particular, the distal end 2b of the tube 2 is configured to receive and pass the suture needle 9 within said curved distal tube portion 2c, wherein the needle 9 is loaded and inserted anteriorly to the hollow tube 2 through the distal end 2b, together with a portion of the suture thread coupled thereto.

According to any of the embodiments of the present invention, said chuck 4 may comprise, at its distal end, a cavity 4a capable of accommodating a needle end, for example a needle base 9b, to provide greater stability during the process of loading and inserting the needle 9 into the curved distal portion 2c.

In other words, the operator can load the needle onto the suture-passing device by simply introducing it within the curved distal tube portion 2c and/or, in embodiments that provide for it, inserting the needle into the cavity 4a. Once the needle 9 is engaged in the cavity 4a, it is recalled within the curved distal tubing portion 2c when the chuck 4 is retracted in the proximal direction according to the manner of use of the device 1 described below.

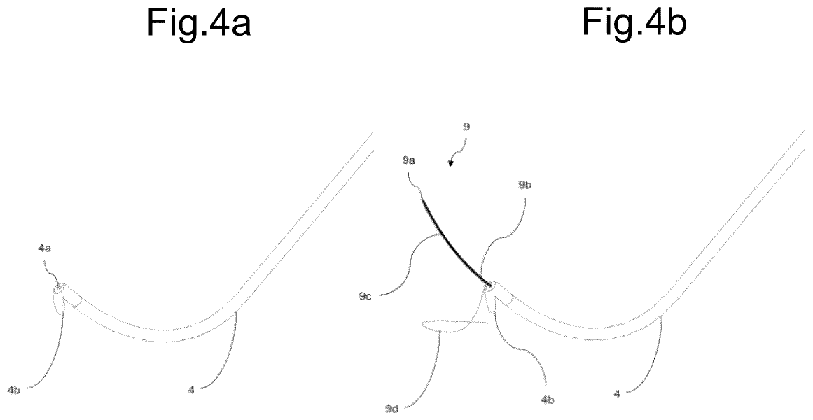

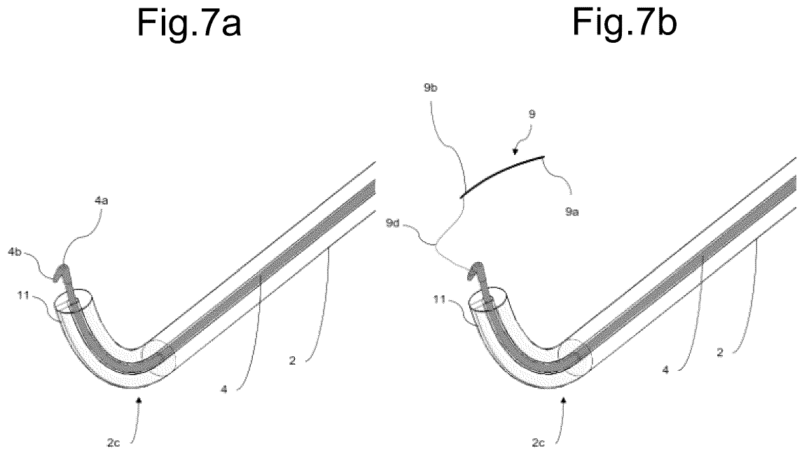

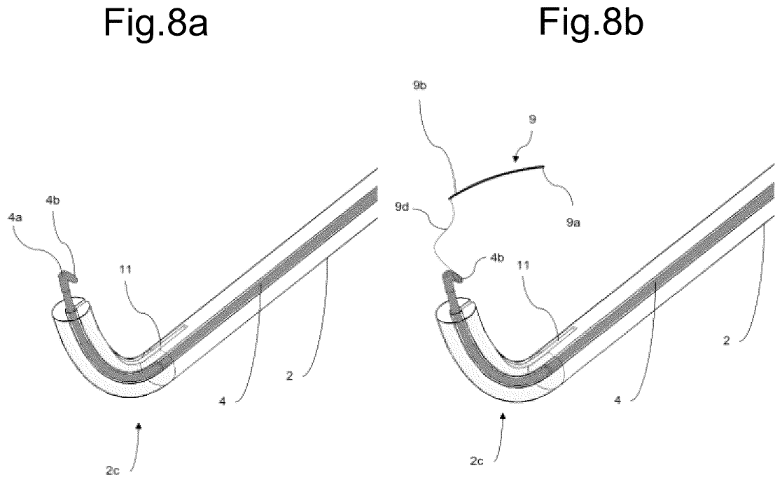

According to an alternative embodiment, as illustrated in FIGS. 4a, 4b, the distal end of the chuck 4 comprises a hook tip 4b. Said hook tip may further comprise, at its distal end, a cavity 4a capable of accommodating a needle base 9b. This embodiment has the advantage of allowing the preloaded suture thread 9d to be wrapped around the suture needle 9 and allowing the operator to wrap the needle thread 9 around said hook 4b so as to facilitate retrieval of the needle 9 during any operative phase. When the chuck 4 is retracted in a proximal direction, it draws the thread in the same direction and allows the retrieval of the needle 9 and facilitates its reinsertion within the distal portion of the tube 2c. According to this embodiment, the distal portion of said hollow tube 2c further comprises a groove 11 formed along the wall of the distal portion of tube 2c and configured to allow the hook tip 4b to travel along the distal portion of tube 2c. The groove may be made on the inner side of the distal portion of tube 2c, i.e. the side having the smaller radius of curvature, as shown in FIGS. 7a, 7b, or on the outer side of the distal portion of tube 2c, i.e. the side having the larger radius of curvature, as shown in FIGS. 8a, 8b. It will be obvious to a person skilled in the art that said groove 11 may be realised along any side of the distal portion of tube 2c. According to a preferred embodiment, the groove 11 is obtained on the outer side, in order to prevent the wire from obstructing the operator's view when using the device. Said hook bit 4b and the groove 11 are dimensioned in such a way as to allow the hook bit 4b to slide along the groove 11 without allowing the chuck 4 to escape from the groove 11.

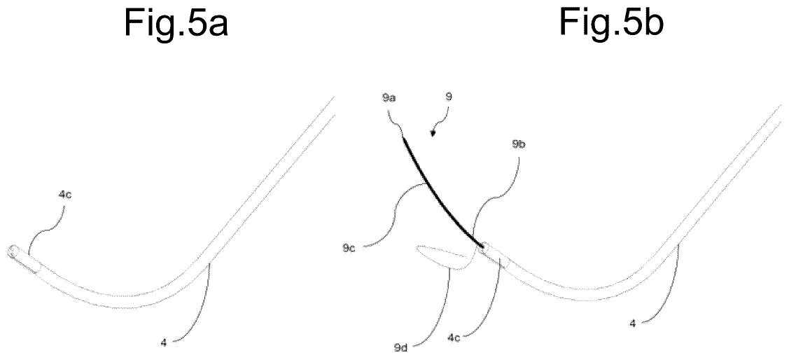

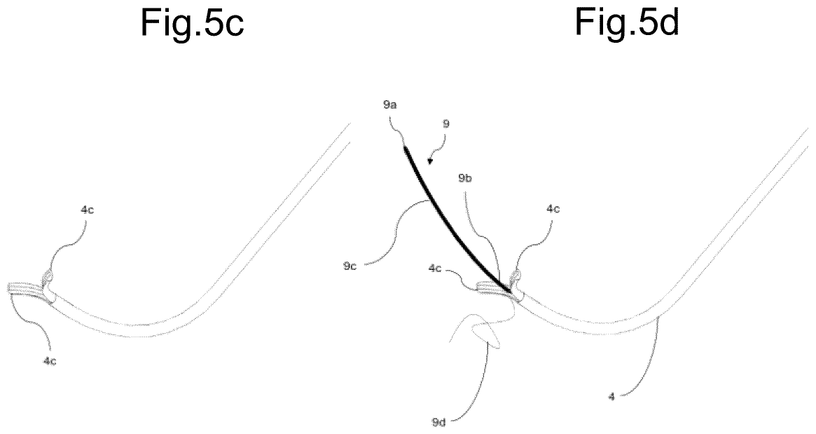

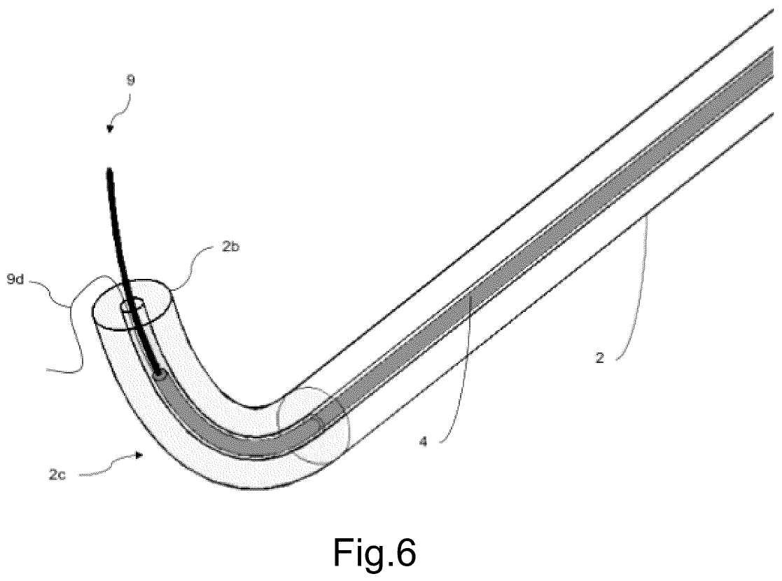

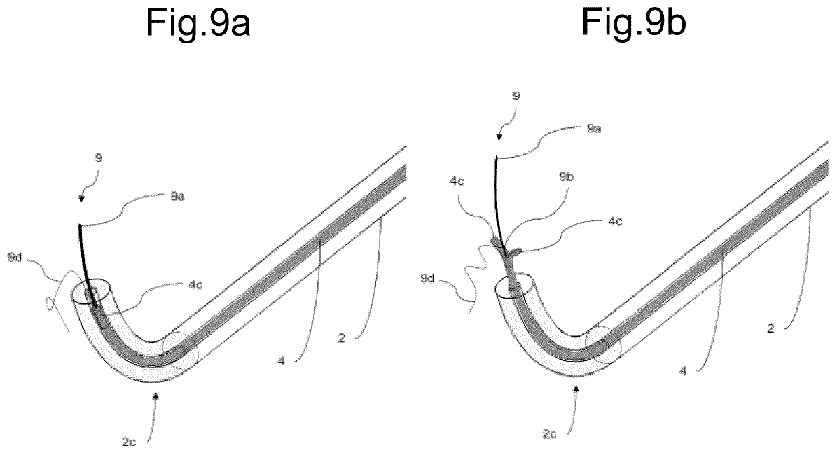

According to another embodiment of the present invention, as shown in FIGS. 5a-5d, 9a and 9b, said chuck 4 may comprise, at its distal end, two or more flaps 4c made of elastic material, preferably with shape memory, and/or configured to assume a closed configuration such as to accommodate a needle end, for example the base of the needle 9b, when the chuck is retracted in a proximal direction within the distal tube portion 2c, and to change to an open configuration when the chuck is pushed in a distal direction towards the outside of the hollow tube 2, so as to release the needle 9. In particular, the embodiment of the flaps 4c made of elastic material is such as to allow the mutual spacing of the distal ends of the flaps 4c in the radial direction when the chuck is pushed outwards from the hollow tube 2, thus in the absence of the spatial constraints determined by the internal walls of the hollow tube 2, and the mutual rapprochement of the distal ends of the flaps 4c when the chuck is retracted internally to the hollow tube 2, wherein the internal walls of the hollow tube 2 keep the flaps 4c in a closed configuration. This embodiment is particularly advantageous for the insertion of the needle 9 within the distal portion of the tube 2c as the flaps 4c, by switching to the closed configuration, prevent inadvertent movement of the needle 9 during reinsertion into the distal portion of the tube 2c.

The handpiece 3 acts as a handle of the device 1 and comprises a housing chamber 3a and an actuator 3b, the latter characterised by a profile geometrically complementary to that of the housing chamber 3a and configured to be inserted and housed within the housing chamber 3a.

The handpiece 3 further comprises a coupling interface 10 at the proximal end of the hollow tube 2, wherein interface 10 refers to the coupling site between the handpiece 3 and the hollow tube 2. Said interface 10 is configured to reversibly connect the proximal end 2a of the hollow tube 2 to the handpiece 3 and allow the rotation of the hollow tube 2 around the actuation axis A. This configuration is advantageous in that the hollow tube 2 is able to rotate about the actuation axis A, allowing the operator to orient the distal end 2b of the tube 3 in a manner consonant with the end use, and in that it allows the decoupling of the tube 2 from the handpiece 3 for replacement and/or maintenance of either component.

Furthermore, the coupling interface 10 can be configured to allow the tube 2 to rotate relative to the handpiece 3 so as to allow the operator to orient the tube with one hand while holding the handpiece 3. This gives the device 1 a further advantage, given the greater freedom and ease for the operator to move the distal end 2b of the tube 3 so as to use the device even on regions of tissue that are difficult to access.

Said coupling between the handpiece 3 and the hollow tube 2 can be realised by any suitable mechanical means known in the state of the art capable of allowing the rotation of the tube 3 around the axis A.

According to a preferred embodiment of the present invention, the coupling interface 10 is characterised by a conical coupling between the handpiece 3 and the proximal end of the hollow tube 2a. In particular, according to this embodiment, the handpiece 3 comprises a conical profile coupling housing chamber 3a and the proximal end 2a of the hollow tube 2 comprises a coupling flange configured to be reversibly coupled to said housing chamber 3a and to allow rotation of the hollow tube 2 about the axis A. According to an alternative embodiment of the present invention, said coupling between the handpiece 3 and the hollow tube 2 can be realised by means of pivotable bearings. According to a further embodiment of the present invention, the handpiece 3 may comprise one or more convex gripping portions configured to facilitate gripping by an operator.

It will be obvious to the person skilled in the art that said handpiece 3 may take various shapes and sizes such as, for example, those of common handles known from the state of the art such as a pistol grip, scissor grip, knob grip or clip grip, provided that the handle allows the operator to operate the piston with one hand, according to the advantage of the present invention described below.

The actuator 3b is preferably a piston defining a proximal end and a distal end, wherein the proximal end of the piston comprises an enlarged base to facilitate gripping and operation with a hand 3c having a diameter greater than the diameter of the piston, and wherein the distal end of the piston is connected to the inner chuck 4. The connection between inner spindle 4 and actuator 3b can be made using any suitable method known from the state of the art, for example by welding, interlocking and/or screwing. Preferably, the connection between the spindle 4 and the actuator 3b is configured to be reversible and allow the removal of the spindle 4. This is advantageous as the described configuration allows the removal of the spindle 4, in case of replacement or maintenance of the component, together with the removal of the hollow tube 2.

According to a preferred embodiment of the present invention, the handpiece 3 comprises a feed-through channel 3d configured to connect said housing chamber 3a with the proximal end 2a of the hollow tube 3 and wherein said inner chuck 4 is connected to the actuator 3b through said feed-through channel 3d.

The actuator 3b is configured to move between a first operating position and a second operating position along said actuating axis A. The internal chuck 4 to the hollow tube 2 is configured to be moved by said handpiece 3 between a first configuration and a second configuration along said tube axis T, depending on the movement of the actuator between said first and second operating positions.

The handpiece 3 also comprises a coaxial spring 5 external to the actuator 3b, arranged within the housing chamber 3a and preferably constrained to the actuator 3b. In other words, the spring 5 surrounds the actuator and, like the latter, remains contained within the housing chamber 3a. Said spring 5 is configured to oppose the movement of the actuator 3b from the first operating position to the second operating position, and to accommodate the movement of the actuator from the second operating position to the first operating position, as further described below.

When the device 1 is not in use, the actuator 3b is in the first operating position, the coaxial spring 5 is extended and the inner chuck 4 is retracted proximally within the distal curved tube portion 2c in the first configuration so as to accommodate said suture needle 9 within said distal curved tube portion 2c.

The operator can hold the device 1 with one hand through the handpiece 3 and, while holding the handle of the device 1, push the actuator 3b in a distal direction along the actuation axis A, for example by pressing the proximal end of the actuator 3b with the thumb. In doing so, the coaxial spring 5 is compressed and the chuck pushed in a distal direction along the tube axis T, such that the chuck extends out of the distal curved tube portion 2c in the second configuration relative to the first configuration so as to push said suture needle completely out of said distal curved tube portion 2c.

The operator can then proceed to couple a suture needle 9 to the chuck by inserting the needle base 9b into said cavity 4a of the distal end of the chuck 4.

Once the suture needle has been coupled to the chuck, the operator can retract the actuator 3b in a proximal direction, for example by releasing the pressure exerted with the thumb or by exerting traction in a proximal direction, thereby returning the chuck 4 to its first configuration and the actuator 3b to its first operating position.

This movement allows the suture needle 9 to be inserted within the curved distal portion 2c of the hollow tube 2, through said distal end 2b of the tube 2.

The distal end 2b is configured to allow the suture needle 9 to be housed in the distal curved portion of the tube 2c when the actuator 3b is in the second operative position. When using the device 1, the operator can grasp the device 1 with one hand through the handpiece 3, locate a tissue region of interest (e.g., a skin wound) and appropriately rotate the hollow tube 2 so that the distal end 2b of the tube 2 allows the suture needle 9, contained in said curved distal portion 2c, to be directed or pointed according to his or her procedural preference.

While holding the handle of the device 1, the actuator 3b is made to move towards the second operative position by applying pressure to the proximal end of the actuator 3b, which pushes the actuator 3b in a distal direction along the actuation axis A, compressing the coaxial spring 5 and pushing the spindle in a distal direction along the tube axis T. Advantageously, the presence of the spring 5 allows the operator fine control of the movement of the actuator 3b during needle outflow 9 and perforation of the tissue region of interest.

The distal end 2b of the tube 2 is further configured to allow the suture needle 9, optionally coupled via the needle base 9b to the cavity 4a of the chuck 4, to exit along an advantageous direction when the suture needle 9 is pushed outwardly by the chuck 4, i.e. when the actuator 3b is in the second operating position and pushes the chuck 4 in a distal direction. For this purpose, the curved distal tube portion 2c is advantageously configured so that, when the curved suture needle is about to exit completely outside the curved distal tube portion 2c, the tip of the curved suture needle is directed either perpendicularly to said longitudinal tube axis T or is at least partially directed along the longitudinal tube axis T towards the handpiece 2.

In this way, the curved profile of the curved distal portion 2c further facilitates the surgical operation in that, when the tip of the curved needle is directed perpendicularly to the longitudinal axis of tube T, the grasping of the needle 9 through, for example, a surgical forceps, is facilitated and facilitates the application of a suture without damaging the surrounding tissues. This constitutes a further advantage over the state of the art, since existing devices do not allow the suture needle to be directed in a direction perpendicular to the longitudinal axis of the tube, but only in a direction substantially parallel to it towards the handpiece, making the procedure of grasping and extracting the needle from the tube in which it is housed difficult for the operator.

If the hollow tube 2 is held coaxial to the handpiece 2, the direction of movement of the actuator 3b and the direction of movement of the spindle 4 are coincident. If, on the other hand, the hollow tube 2 is oriented with respect to the handpiece and the actuation axis A, the direction of movement of the spindle along the T-axis is rotated with respect to the A-axis by an angle equal to the angle of the orientation. Advantageously, the flexible nature of spindle 4 ensures that the thrust of actuator 3b is transmitted effectively to it. When the actuator 3b is in the second operating position, the coaxial spring 5 is compressed again and the inner spindle 4 extends out of the curved distal tube portion 2c in the second configuration from the first configuration. At this stage, the suture needle 9 is pushed from the chuck 4 outwards from the housing defined by the curved distal tube portion 2c and can penetrate the biological tissue of interest.

At this stage, the operator may use one or more additional medical devices, such as medical forceps, to grasp the suture needle 9, disengage it from the chuck 4 and proceed to apply a suture.

Following this, the operator may decide to re-insert the suture needle 9 within the distal curved portion 2c of the hollow tube 2 in a similar manner as described for the non-use phase of the device 1 and retract the actuator 3b returning it to its first operating position.

Consequently, the spindle 4 will also be retracted to the first configuration. The procedure described above can then be repeated as many times as the number of suture points to be applied.

One of the advantages of the suture-passer and needle-pusher device according to the present invention lies in the facilitation of the procedures of loading the needle 9 into the tube 2 and inserting the suture needle 9 by the operator using only one hand.

In order to ensure the integrity of the device 1, to prevent inadvertent movements of the actuator 3b during use by the operator, and/or to further facilitate needle loading, the device 1 may comprise at least one safety system, among those best described below. The handpiece 3 comprises a first retaining means 6 configured to prevent the actuator 3b from discharging in a proximal direction from the housing chamber 2a. In particular, the first retaining means 6 prevents over-extension of the spring 5 and thus further movement of the actuator 3b in the proximal direction when it is already in the first operating position.

Said first retaining means 6 comprises at least one shaped protrusion 6a located in said housing chamber 3a of the actuator and at least one shaped protrusion 6b located on said actuator 3b, said two shaped protrusions 6a, 6b being configured to create mechanical interference with each other to prevent the relaxation of the spring 5 from excessively pushing the actuator 3b in a proximal direction out of the housing chamber 3a when the actuator 3b is in the first or, possibly, in the second operating position. The presence of one or more shaped protrusions prevents the actuator 3b from protruding out of the housing chamber 3a by the action of the spring 5 even when the device 1 is not in use. According to any one embodiments of the present invention, the handpiece 3 also comprises at least a second retaining means 7 configured to bind the actuator 3b to the handpiece 3 and to prevent unintentional movement of the actuator 3b in a proximal direction when in the second operating position. In particular, the second retaining means 7 comprises a through-hole 7c and a pin 7a constrained to the enlarged base 3c of the actuator 3b and configured to slide through the through-hole 7c.

The pin 7a includes a hooked end 7b configured to engage the handpiece 3 and reversibly bind the actuator 3b to the handpiece 3 when the actuator 3b is in the second operating position.

The hooked end 7b is movable to disengage the handpiece 3 and allow the actuator 3b to move in a proximal direction towards the first operating position. The disengagement of the hooked end 7b can be achieved by applying a bending force to the pin 7a, which allows the pin 7a to slide back through the through-hole 7c.

The use of the second detent means 7 prevents the actuator 3b from suddenly returning to the first operating position due to the action of the spring 5 while in the second operating position.

This configuration is advantageous in that it facilitates the process of loading and inserting the needle 9 into the curved distal portion 2c. Once the actuator 3b has been constrained to the handpiece 3 by the second detent means 7, the operator has both hands available to proceed with the coupling of the needle 9 to the chuck 4 since he does not have to maintain the pressure exerted on the actuator 3b to bring it into the second operating position. Similarly, the same advantage occurs when applying sutures.

It is also an object of the present invention to provide a method of suturing comprising one or more of the steps of the above-described process using a suture-passing device 1 according to any of the embodiments described and forming part of the present disclosure.

The present invention has been described herein with reference to preferred embodiments thereof. It is to be understood that other forms of embodiment may exist or be contemplated which share the same inventive core with the one described herein, all of which fall within the scope of protection of the claims set forth below.

For example, a suture-passer and/or needle-pusher device adapted to handle more than one suture thread and/or more than one needle, e.g., two, three or four suture threads and/or needles, is within the scope of the present disclosure.

According to a further embodiment of the present invention, not shown in the figures, the device is characterised by two hollow tubes (2, 2′) and two chucks (4, 4′) inside the hollow tubes, such as to manage the loading and unloading of two suture needles according to the same methods and characteristics described in the present disclosure, wherein the two hollow tubes are coupled to the same handpiece and the two chucks are connected to the same actuator.

1. A suture passer and needle pusher device comprising:
a hollow tube having a straight proximal tube portion defining a longitudinal axis T of the tube and having a proximal end, and a curved distal tube portion extending from said straight proximal tube portion up to a distal end of the hollow tube, the distal end being configured to receive and pass a suture needle into said curved distal tube portion, a handpiece coupled to the proximal end of the hollow tube, where the handpiece defines an actuation axis A and comprises a housing chamber and an actuator, the actuator being housed inside said housing chamber and being configured to move between a first operating position and a second operating position along said actuation axis A;
a chuck inside the hollow tube and connected to the actuator, the chuck being configured to be moved by said handpiece between a first configuration and a second configuration;
wherein:
said curved portion of distal tube describes an arc of circumference subtended by a first angle in the center of not less than 45° and is configured to accommodate inside a curved suture needle also describing a second arc of circumference no longer of said first arc of circumference, said second arc of circumference having the same radius as said first arc of circumference;
the actuator also includes a coaxial spring, said coaxial spring being constrained to the actuator and configured to oppose the movement of the actuator from the first operating position to the second operating position and to support the movement of the actuator from the second operating position to the first operating position, such that when the actuator is in the first operating position, the coaxial spring is extended and the internal chuck is retracted proximally to the inside of the curved distal tube portion in the first configuration so as to fully accommodate said suture needle inside said curved distal portion, and when the actuator is in the second operative position, the coaxial spring is compressed and the internal chuck extends out of the curved distal tube portion in the second configuration with respect to the first configuration so as to push said suture needle completely out of the said curved distal portion, and
wherein said curved distal tube portion is configured so that, when the curved suture needle is about to exit completely outside the curved distal tube portion, the tip of the curved suture needle is directed either perpendicularly to said longitudinal axis of tube T or is at least partially directed along the longitudinal axis of tube T towards said handpiece.

2. The device according to claim 1, wherein the distal end of the chuck comprises a hook tip and wherein the curved distal tube portion comprises a groove, said groove being formed along the wall of the distal portion of the tube and configured to allow the run of the hook tip along the distal portion of the tube.

3. The device according to claim 2, wherein the groove is formed on a side of the hollow tube having a greater bending radius or on a side of the hollow tube having a smaller bending radius.

4. The device according to claim 1, wherein the chuck comprises, at its distal end, a cavity adapted for receiving a needle end.

5. The device according to claim 1, wherein the distal end of the chuck 4 comprises two or more flaps made of elastic material and configured to assume a closed configuration such as to accommodate a needle end when the chuck is retracted proximally into the curved distal tube portion and to change to an open configuration when the chuck is pushed distally outward of the hollow tube, thereby freeing the needle.

6. The device according to claim 1, wherein said portion of curved distal tube describes an arc of circumference subtended by a first angle at the center of not less than 60°, preferably wherein said curved portion of distal tube describes an arc of circumference subtended by a first angle at the center of not less than 90°, so that, when the curved suture needle is about to come out completely outside the curved portion of distal tube, the tip of the curved suture needle is directed parallel to said longitudinal axis of tube T towards said handpiece.

7. The device according to claim 1, wherein the handpiece comprises a first stop means, the first stop means being configured to prevent the actuator from coming out proximally from the housing chamber.

8. The device according to claim 7, wherein said first stop means comprises a first shaped protrusion located in said housing chamber, and a second shaped protrusion located on said actuator, the first and second shaped protrusions being configured to create mutual mechanical interference.

9. The device according to claim 1, where the actuator is a piston defining a proximal end and a distal end, where the proximal end of the piston comprises an enlarged base having a diameter greater than the piston diameter.

10. The device according to claim 1, wherein said handpiece comprises a second stop means, the second stop means being configured to constrain the actuator to the handpiece and to prevent movement of the actuator in the proximal direction in the second operating position.

11. The device according to claim 10, where the handpiece of the device comprises a through hole and where the second stop means comprises a pin constrained to said enlarged base of the actuator and configured to slide through said through hole, the pin comprising a hooked end configured to engage the handpiece and reversibly constrain the actuator to said handpiece when the actuator is in the second operating position, said hooked end being movable to disengage the handpiece and allow the actuator to move towards the first operating position.

12. The device according to claim 1, wherein the bandpiece comprises a coupling interface at the proximal end of the hollow tube, the interface being configured to connect in the proximal end of the hollow tube to the handpiece and allow the rotation of the hollow tube around the actuation axis A.

13. The device according to claim 12, wherein the connection interface of the handpiece to said hollow tube comprises a conical coupling between the handpiece and the proximal end of the hollow tube.

14. The device according to claim 1, in which the curved distal portion is made of plastically deformable material.

15. The device according to claim 1, comprising a suture needle, the suture needle being constituted by a curved body and defining two ends, said two ends being a piercing tip and a needle base.
